(19) United States
(12) Patent Application Publication
(10) Pub. No.: US 2005/0177005 A1
(43) Pub. Date: Aug. 11, 2005

(54) Method for producing polyether alcohols

(75) Inventors: Raimund Ruppel, Dresden (DE); Eva Baum, Schwarzheide (DE); Jurgen Winkler, Schwarzheide (DE); Edward Bohres, Mannheim (DE)

Correspondence Address: OBLON, SPIVAK, MCCLELLAND, MAIER & NEUSTADT, P.C., 1940 DUKE STREET, ALEXANDRIA, VA, 22314, US

(73) Assignee: BASF AKTIENGESELLSCHAFT, LUDWIGSHAFEN (DE)

(21) Appl. No.: 10/515,350

(22) PCT Filed: Jun. 6, 2003
(86) PCT No.: PCT/EP03/05936

(30) Foreign Application Priority Data
Jun. 13, 2002 (DE) 102 26 415.5

(51) Int. Cl. C07C 41/03

(52) U.S. Cl. 568/679

(57) ABSTRACT

The invention relates to a method for producing polyether alcohols, comprising the following steps: a) reacting a low-molecular alcohol with alkylene oxide in the presence of a basic catalyst in order to form an alkylene oxide addition product having a molecular weight ranging from 200 to 900 g/mol; b) separating the basic catalyst from the product obtained in step a), and; c) reacting the product obtained in step b) with another alkylene oxide in order to produce the desired final product while using at least one DMC catalyst. The inventive method is characterized in that sodium hydroxide is used as the basic catalyst in step a).

The present invention relates to polyether alcohols, to an improved process for preparing them by means of DMC catalysis and also to their use, in particular for preparing polyurethanes.

Polyurethanes are prepared in large quantities. They are usually prepared by reacting polyisocyanates with compounds having at least two hydrogen atoms which are reactive toward isocyanate groups, in particular polyether alcohols and/or polyester alcohols.

Polyester alcohols are usually prepared by reacting polyfunctional alcohols with polyfunctional carboxylic acids.

Polyether alcohols are usually prepared by catalytic addition of alkylene oxides onto H-functional starter substances, in particular alcohols and/or amines. As catalysts, use is usually made in industry of alkaline substances, preferably alkali metal hydroxides, with potassium hydroxide having the greatest industrial importance.

Recently, multimetal cyanide catalysts, frequently also referred to as DMC catalysts, have gained increasing importance. The advantages of the DMC catalysts are that the addition reaction of the alkylene oxides proceeds at a higher reaction rate and that the polyether alcohols prepared in this way have a lower content of unsaturated units in the polyether chain. The DMC catalysts can be employed particularly advantageously in the preparation of polyether alcohols having a high molecular weight and a low functionality, as are used, in particular, for producing flexible polyurethane foams. To produce rigid polyurethane foams, use is usually made of polyether alcohols having a high functionality, in particular those having a functionality of at least 4. The starter substances used for preparing such polyether alcohols are usually solid. Such solid starter substances have hitherto not yet been able to be reacted with alkylene oxides by means of DMC catalysts.

A further disadvantage of the use of DMC catalysts is the difficulty of starting the reaction. Thus, it is very difficult to react low molecular weight alcohols such as glycerol with alkylene oxides in the presence of DMC catalysts. The low molecular weight alcohols are usually initially reacted with alkylene oxides in the presence of other catalysts to form an intermediate which is purified if necessary and then reacted with further alkylene oxide in the presence of DMC catalysts to form the desired polyether alcohol. The most widespread process for preparing polyether alcohols and also intermediates suitable as starter compounds for DMC catalysis comprises addition of alkylene oxides onto the abovementioned low molecular weight starter alcohols in the presence of potassium hydroxide as catalyst with subsequent neutralization and removal of the potassium salts formed, e.g. by filtration. If the intermediates prepared in this way are to be used as starter compounds for the DMC-catalyzed addition of further alkylene oxide, the polyetherols have to be purified very carefully, possibly in a further process step, to remove the potassium salts and other alkaline constituents virtually completely. An excessive increase in the concentration of H⁺ ions is likewise disadvantageous and thus has to be avoided. This procedure is very complicated and therefore has an adverse effect on the economics of the process.

Furthermore, if polyether alcohols are to be prepared by means of alkali metal hydroxides and DMC catalysts in one and the same production plant, it is generally necessary to employ laborious flushing processes when changing from KOH to DMC, and it cannot be ensured that the subsequent DMC-catalyzed processes will proceed in a trouble-free manner and give an in-specification product. Apart from the additional economic disadvantages, this can also lead to safety problems since undefined reaction conditions with accumulated alkylene oxide can arise in this way.

It is an object of the present invention to provide a process for preparing polyether alcohols which can be prepared by addition of alkylene oxides onto H-functional starter substances by means of DMC catalysts, with the starter substances onto which alkylene oxides are to be added by means of DMC catalysts being able to be produced in a simple and reproducible manner. It should also be ensured that polyether alcohols can be prepared by means of alkaline and DMC catalysts in the same production plant without complicated cleaning steps being necessary when changing catalyst.

We have found that this object is achieved by using sodium hydroxide as catalyst in the preparation of the starter compound for the DMC-catalyzed addition reaction of alkylene oxides.

The present invention accordingly provides a process for preparing polyether alcohols, which comprises the steps
a) reacting a low molecular weight alcohol with alkylene oxide in the presence of a basic catalyst to form an alkylene oxide addition product having a molecular weight in the range from 200 to 1 500 g/mol, preferably from 200 to 900 g/mol,
b) separating the basic catalyst from the product from step a), and
c) reacting the product from step b) with further alkylene oxide in the presence of at least one DMC catalyst to form the desired end product,
   wherein the basic catalyst used in step a) is sodium hydroxide.

The invention also provides polyether alcohols which can be prepared by the process of the present invention and provides for the use of the polyether alcohols of the present invention, in particular for preparing polyurethanes.

Furthermore, the present invention provides a process for preparing polyurethanes by reacting
d) polyisocyanates with
e) compounds having at least two hydrogen atoms which are reactive toward isocyanate groups, wherein at least one polyether alcohol according to the present invention is used as compounds having at least two active hydrogen atoms.

As low molecular weight alcohols in process step a), use is made, in particular, of alcohols having from 2 to 8 hydroxyl groups, preferably aliphatic and cycloaliphatic alcohols having from 2 to 8 carbon atoms in the branched or unbranched alkyl chain or in the cycloaliphatic skeleton. In particular, the polyfunctional alcohols are selected from the group consisting of glycerol, trimethylolpropane, pentaerythritol, dipentaerythritol and tripentaerythritol, sorbitol, sucrose, ethylene glycol and its homologues, in particular ethylene glycol and/or diethylene glycol, propylene glycol and its higher homologues, in particular propylene glycol and/or dipropylene glycol, 1,3-propanediol, 1,2-, 1,3-, 2,3- and 1,4-butanediol, pentanediols and hexanediols, in particular 1,5-pentanediol and 1,6-hexanediol.

Further possible starting materials for process step a) are hydrolyzed starch, glucose syrup, ethanol, propanols, hydroxycarboxylic acids, hydroxyaldehydes and hydroxyketones, as long as no functional groups which are attacked by the sodium hydroxide, for example ester groups, or which interfere in the subsequent DMC-catalyzed addition step, for example amino groups, are present in the molecule.

Step a) is preferably carried out so that the degree of polymerization remains as small as possible so as to make optimum use of the effectiveness of the subsequent DMC-catalyzed addition reaction. The end product from step a) should accordingly have a very low mean molecular weight, depending on the functionality and the starter alcohol used. Step a) is preferably carried out to a molecular weight of the product in the range from 200 to 1 500 g/mol, preferably from 200 to 900 g/mol.

The amount of sodium hydroxide used in step a) is preferably in a range from 0.05 to 2% by weight, preferably from 0.1 to 1% by weight, based on the amount of the intermediate prepared in step a).

The reaction in process step a) can proceed under the conditions customary for the preparation of polyether alcohols, as are described, for example, in the Kunststoff-Handbuch, Volume 7 “Polyurethane”, edited by Günter Oertel, Carl-Hanser-Verlag, Munich, Vienna 1993, pages 63 to 65.

The reaction of the low molecular weight alcohols with the alkylene oxides in step a) preferably takes place at pressures in the range from 1 to 20 bar, in particular from 2 to 10 bar, and temperatures in the range from 60 to 150° C., in particular from 80 to 130° C. Furthermore, the preparation is preferably carried out under a protective gas atmosphere, in particular under a nitrogen and/or argon atmosphere.

As alkylene oxides, it is possible to use, for example, ethylene oxide, propylene oxide, butylene oxide, isobutylene oxide and any mixtures thereof. Preference is given to using ethylene oxide, propylene oxide and mixtures thereof.

In step a), the alkylene oxides can be used individually in the form of blocks and, when using more than two different alkylene oxides, can be added on in any mixing ratio as mixed blocks. Furthermore, the mixing ratio of the alkylene oxides during their addition can be varied either discontinuously or continuously.

After addition of the alkylene oxides is complete, an after-reaction phase is usually provided to achieve complete reaction of the alkylene oxides. This is usually followed by work-up of the product from step a). Here, secondary components such as unreacted monomers, volatile compounds and by-products can be removed by various methods known to those skilled in the art, for example by distillation, work-up by means of a thin film evaporator, nitrogen stripping and/or steam stripping.

The intermediate from process step a) is treated in step b) with water and a neutralizing agent using the customary technologies known to those skilled in the art to remove the catalyst. Here, preference is given to using acids, in particular phosphoric acid, but carbon dioxide or acid ion exchangers, for example Ambosol®, can also be used. The neutralization is usually followed by removal of the sodium salts formed or of the ion exchanger using customary technologies known to those skilled in the art, e.g. filtration or centrifugation.

The neutralization can, if desired, be carried out in the same reactor system used in step a), which further simplifies the subsequent cleaning for carrying out the addition reaction of the alkylene oxides in step c).

Surprisingly, the removal of the basic catalyst in step b) does not have to be carried out to such low contents as in the case of the customary use of potassium hydroxide which must be separated off to a residual content of potassium ions of less than 5 ppm, preferably less than 3 ppm.

The worked-up intermediate from step b) preferably has a residual alkalinity of less than 15 ppm, particularly preferably less than 10 ppm, in particular in the range from 5 to 30, measured by means of titration. At an alkalinity in this range attributable to potassium ions, the reaction with alkylene oxides using DMC catalysts would proceed only with difficulty, for example with a long induction period or with use of increased amounts of DMC catalyst.

More thorough removal of sodium salts, for example to a content of less than 5 ppm, is not necessary. However, for particular applications of the process of the present invention, for example when using very sensitive DMC catalysts in step c), a further reduction in the alkalinity can be advantageous. In any case, however, use of potassium hydroxide as catalyst would require removal to lower residual contents.

As a result, step b) can proceed significantly more simply. This is advantageous, in particular, when using highly viscous or solid starter substances, since the intermediates from process step a) usually have a relatively high viscosity in the case of such compounds and are therefore difficult to filter.

A further advantage of the process of the present invention is the possible simplification of the way in which the process is carried out. If the addition reaction of further alkylene oxide using a DMC catalyst in step c) is to be carried out immediately after step b) in the same reactor system, cleaning the reactor once or twice with water is generally sufficient in the process of the present invention. If potassium hydroxide were to be used in place of sodium hydroxide in step a), the reactor would generally have to be flushed with water a greater number of times and a number of test syntheses using the DMC catalyst with frequently nonreproducible start-up behavior and undesirable product properties, for example a high viscosity or a broad molecular weight distribution, might be necessary.

To carry out step c), the product obtained after step b), which, as described, preferably has a molecular weight in the range from 200 to 1 500 g/mol, more preferably from 200 to 900 g/mol, and a hydroxyl number in the range from 150 to 800 mg KOH/g, is admixed with DMC catalyst and then reacted with alkylene oxides to give the finished polyether alcohol. In general, from 10 to 500 ppm of DMC catalyst, preferably from 15 to 200 ppm of DMC catalyst, particularly preferably from 25 to 150 ppm of DMC catalyst, in each case based on the mass of the expected end product, are used. Surprisingly, no decreases in activity occur even when the amounts of catalyst are small.

The DMC catalyst can be used as a powder or in the form of a suspension, preferably in a polyol as described, for example, in EP 090 444.

The preparation of the polyether alcohols of the present invention is carried out, as stated above, by addition of alkylene oxides onto the polyether alcohols from step b) in the presence of multimetal cyanide catalysts as per step c).

As alkylene oxides, it is possible to use, for example, ethylene oxide, 1,2-propylene oxide, 1,2-butylene oxide, 1,2-isobutylene oxide and any mixtures thereof. Preference is given to using ethylene oxide and 1,2-propylene oxide and also mixtures thereof.

In step c), too, the alkylene oxides can be added on individually in the form of blocks and, when using more than two different alkylene oxides, can be added on in any mixing ratio as mixed blocks. Furthermore, the mixing ratio of the alkylene oxides can be varied either discontinuously or continuously during their addition. After activation of the starting mixture in process step c) is complete, it is possible, if desired, for further alcohol, either the same alcohol as in step a) or a different alcohol, to be metered in together with the alkylene oxide, as is described, for example, in DD 203734 or EP 879 259.

The structure of the polyether chain depends on the use to which the polyether alcohols are to be put. In the case of polyether alcohols which are to be used for flexible polyurethane slabstock foams, preference is given to adding on a block consisting essentially, preferably entirely, of propylene oxide at the end of the chain. In the case of polyether alcohols which are to be used for molded flexible polyurethane foams, preference is given to adding on a block consisting essentially, in particular entirely, of ethylene oxide at the end of the chain. This block can be added on by metering in pure ethylene oxide.

In a preferred embodiment of the process of the present invention, the alkylene oxides are metered in in such a way that the ratio of the alkylene oxides in the mixture changes during the course of the addition. Thus, an end block consisting of ethylene oxide units can be added on by reducing the propylene oxide content of a mixture of ethylene oxide and propylene oxide being introduced until only pure ethylene oxide is being added on at the end of the introduction of alkylene oxide. This type of metered addition is described in WO 01/44347.

A further possible way of carrying out the addition reaction of the alkylene oxides in step c) is described in WO 99/51661. In this method, not more than 5% of propylene oxide is added on in the absence of ethylene oxide in order to reduce the content of very high molecular weight constituents.

Multimetal cyanide catalysts used are usually compounds of the formula (I),
M¹ₐ[M²(CN)_{b}(A)_{c}]_{d}.fM¹gXₙ.h(H₂O).eL (I),
where
M¹ is a metal ion selected from the group consisting of Zn2+, Fe2+, Co3+, Ni2+, Mn2+, Co2+, Sn2+, Pb2+, Mo4+, Mo6+, Al3+, V4+, V5+, Sr2+, W4+, W6+, Cr2+, Cr3+, Cd2+,
M² is a metal ion selected from the group consisting of Fe2+, Fe3+, Co2+, Co3+, Mn2+, Mn3+, V4+, V5+, Cr2+, Cr3+, Rh3+, Ru2+, Ir3+

and M¹ and M² are identical or different,
A is an anion selected from the group consisting of halide, hydroxide, sulfate, carbonate, cyanide, thiocyanate, isocyanate, cyanate, carboxylate, oxalate and nitrate,
X is an anion selected from the group consisting of halide, hydroxide, sulfate, carbonate, cyanide, thiocyanate, isocyanate, cyanate, carboxylate, oxalate and nitrate,
L is a water-miscible ligand selected from the group consisting of alcohols, aldehydes, ketones, ethers, polyethers, esters, ureas, amides, nitriles and sulfides,
   and

a, b, c, d, g and n are chosen so that the compound is electrically neutral and
e is the coordination number of the ligand or zero,
f is a fraction or integer greater than or equal to 0 and
h is a fraction or an integer greater than or equal to 0.

Such compounds are generally known. They are prepared by known methods, for example as described in EP 862 947, by combining the aqueous solution of a water-soluble metal salt with the aqueous solution of a hexacyanometalate compound, in particular a salt or an acid, and, if required, a water-soluble ligand is added thereto either during or after the two solutions have been combined.

To prepare the polyether alcohols of the present invention, the compounds from step b) are firstly mixed with the DMC catalyst and the alkylene oxide or the mixture of alkylene oxides is introduced into this mixture. If appropriate, solvents such as toluene, xylene, tetrahydrofuran, acetone, 2-methylpentanone, cyclohexanone or others can be added to the reaction mixture.

Surprisingly, the reaction of the reaction products from step b) with the alkylene oxides in the presence of the multimetal cyanide catalysts starts reproducibly without problems and without the occurrence of a prolonged induction period.

Even at relatively high molecular weights, the reaction proceeds without an undesirable increase in viscosity, from which it can be concluded that there is reduced formation of undesirable very high molecular weight fractions in the polyether alcohol.

Step c) is preferably carried out at pressures in the range from 1 to 20 bar, in particular from 2 to 10 bar, and temperatures in the range from 60 to 150° C., in particular from 80 to 130° C. Furthermore, the reaction is, for safety reasons, usually carried out under a protective gas atmosphere, in particular a nitrogen and/or argon atmosphere. After the introduction of alkylene oxides is complete, after-reaction time is usually provided to achieve complete reaction of the alkylene oxides. This is followed by the work-up of the polyetherols. Secondary components such as unreacted monomers, volatile compounds and by-products can be removed by various methods known to those skilled in the art, for example by distillation, work-up by means of a thin film evaporator, nitrogen stripping and/or steam stripping.

If appropriate, suspended material and solids can be removed from the reaction mixture by various methods known to those skilled in the art, for example centrifugation or filtration.

After the preparation, the DMC catalyst can in principle remain in the polyether alcohol. If required for particular applications, it can also be partly or completely removed, for example by means of filtration. After the preparation, additives such as antioxidants or stabilizers are usually added to the polyether alcohols.

The polyether alcohols of the present invention preferably have an average functionality of at least 2, preferably in the range from 2 to 8, in particular from 2 to 5, a hydroxyl number in the range from 20 to 600 mg KOH/g and a viscosity determined in accordance with DIN 53 015 at 25° C. in the range from 50 to 5 000 mPas.

The polyether alcohols of the present invention are preferably used for preparing polyurethanes. The preparation of the polyurethanes is carried out according to methods known per se by reaction of the polyols e) with polyisocyanates d). Depending on the desired properties of the polyurethanes, it is possible to use the polyether alcohols of the present invention either alone or together with other compounds having at least two hydrogen atoms which are reactive toward isocyanate groups. Compounds which have at least two hydrogen atoms capable of reaction with isocyanate groups and can be used together with the polyether alcohols of the present invention for the reaction with polyisocyanates include polyether alcohols, polyester alcohols and also, if desired, bifunctional or polyfunctional alcohols and amines having a molecular weight in the range from 62 to 1 000 g/mol, known as chain extenders and crosslinkers. Furthermore, catalysts, blowing agents and the customary auxiliaries and/or additives may be used.

As stated above, the process of the present invention makes it possible for polyether alcohols which are based on solid or highly viscous starters and whose preparation by means of DMC catalysis has hitherto been difficult to be prepared particularly advantageously. A further advantage of the process of the present invention is the simple work-up of the prepolymer which is reacted in the presence of DMC catalysts to give the polyether alcohol.

Such compounds and processes for preparing polyurethanes are described, for example, in the Kunststoff-Handbuch, Volume VII, “Polyurethane” 3rd Edition 1993, edited by Dr. G. Oertel (Carl-Hanser-Verlag, Munich).

The polyether alcohols prepared by the process of the present invention can be used, for example, for producing polyurethane foams, sealing compositions, coatings or as crosslinkers.

The invention is illustrated by the following examples.

### EXAMPLE 1

### Preparation of the Starter Compound using Sodium Hydroxide as Catalyst

In a clean and dry 20 l stirrer tank reactor, 2 730 g of glycerol were admixed with 45 g of solid sodium hydroxide and heated to 120° C. while stirring. At this temperature, the water of reaction formed was subsequently distilled off at a pressure of about 40 mbar until a water content of <0.1% had been reached. After addition of 630 g of ethylene glycol, 16 670 g of propylene oxide were metered in over a period of about 5 hours. After the metered addition was complete, the mixture was allowed to react further until the pressure was constant. Volatile constituents were then distilled off from the reaction mixture.

The contents of the reactor were subsequently admixed with 1 000 g of water and 123 g of 85% strength phosphoric acid, stirred at 90° C. for 60 minutes and subsequently distilled at 15 mbar until the water content was 0.05%. The product was drained from the reactor and filtered through a Seitz deep bed filter T 500.

The colorless polyether alcohol obtained had the following properties: OH number: 306 mg KOH/g, acid number: 0.102 mg KOH/g, water content: 0.077%, viscosity (25° C.): 215 mPas, alkalinity: 6.1 ppm of K.

After the product had been drained from the reactor, the reactor was rinsed twice with water and dried and used in this state for subsequent syntheses.

### EXAMPLE 2

### Preparation of the Polyether Alcohol by Means of DMC Catalysis

3 200 g of the intermediate from example 1 were admixed in the same 20 l stirred tank reactor with 44 g of a 4.53% strength suspension of a DMC catalyst prepared as described in example 1 of EP 862 947, which corresponds to 100 ppm of DMC catalyst based on the product to be prepared, and dewatered at 120° C. and a pressure of about 40 mbar until the water content was below 0.02%. About 400 g of propylene oxide were subsequently introduced and commencement of the reaction was awaited. This could be recognized by a brief temperature increase and a rapid drop in the pressure in the reactor. After the reaction had started, 16 450 g of a mixture of 14 910 g of propylene oxide and 1 940 g of ethylene oxide were subsequently metered in at the same temperature over a period of about 2.5 hours. After a constant pressure in the reactor had been reached, unreacted monomers and other volatile constituents were distilled off under reduced pressure and the product was drained from the reactor. The colorless polyether alcohol obtained had the following properties: OH number: 49.4 mg KOH/g, acid number: 0.033 mg KOH/g, water content: 0.011%, viscosity (25° C.): 543 mPas, M_{w}=3 025 g/mol, D=1.381.

1. A process for preparing polyether alcohols, which comprises the steps
a) reacting a low molecular weight alcohol with alkylene oxide in the presence of a basic catalyst to form an alkylene oxide addition product having a molecular weight in the range from 200 to 900 g/mol,
b) separating the basic catalyst from the product from step a), and
c) reacting the product from step b with further alkylene oxide in the presence of at least one DMC catalyst to form the desired end product,
wherein the basic catalyst used in step a) is sodium hydroxide and the product from step b) has a sodium ion content of from 5 to 30 ppm.

2. A process as claimed in claim 1, wherein the molecular weight of the product from step a) is in the range from 200 to 900 g/mol.

3. A process as claimed in claim 1, wherein the low molecular weight alcohol used in step a) is an alcohol having a functionality of from 1 to 8.

4. A process as claimed in claim 1, wherein the low molecular weight alcohol in step a) is selected from the group consisting of glycerol, trimethylolpropane, pentaerythritol, dipentaerythritol and tripentaerythritol, sorbitol, sucrose, ethylene glycol and its homologues, in particular ethylene glycol and/or diethylene glycol, propylene glycol and its higher homologues, in particular propylene glycol and/or dipropylene glycol, 1,3-propanediol, 1,2-, 1,3-, 2,3- and 1,4-butanediol, pentanediols and hexanediols.

5. A process as claimed in claim 1, wherein the polyether alcohol after step c) has a hydroxyl number in the range from 20 to 600 mg KOH/g.

6. A polyether alcohol which can be prepared by a process as claimed in claim 1.

7. A process for preparing polyurethanes by reacting
d) polyisocyanates with
e) compounds having at least two hydrogen atoms which are reactive toward isocyanate groups,
wherein at least one polyether alcohol as claimed in claim 6 is used as compounds having at least two hydrogen atoms which are reactive toward isocyanate groups.

8. A polyurethane which can be prepared by a process as claimed in claim 7.
